(19) United States
(12) Patent Application Publication
(10) Pub. No.: US 2005/0216039 A1
(43) Pub. Date: Sep. 29, 2005

(54) Method and device for catheter based repair of cardiac valves

(76) Inventor: Robert J. Lederman, Chevy Chase, MD (US)

Correspondence Address: BERENATO, WHITE & STAVISH, LLC, 6550 ROCK SPRING DRIVE, SUITE 240, BETHESDA, MD, 20817, US

(21) Appl. No.: 11/127,112

(22) Filed: May 12, 2005

Related U.S. Application Data
(63) Continuation of application No. PCT/US03/36617, filed on Nov. 14, 2003.
(60) Provisional application No. 60/426,984, filed on Nov. 15, 2002.

(51) Int. Cl. A61F 2/24; A61B 17/04; A61B 17/12

(52) U.S. Cl. 606/144; 623/2.1

(57) ABSTRACT

Disclosed is a system and method for catheter-based repair of cardiac valves, including transcatheter-mitral-valve-cerclage annuloplasty and transcatheter-mitral-valve reapposition. An exemplary embodiment of the system includes: a guiding cathether; one or more secondary catheters, such as a valve-manipulation catheter and one or more optional suture-clip-pledget assemblies; and/or a canalization-needle catheter. Imaging methods and devices can be used to assist the operator of the system in determining the placement and orientation of the system within a subject's body. One exemplary imaging method is real-time magnetic-resonance imaging.

### CROSS-REFERENCE TO RELATED APPLICATION

This application is a continuation of International Application PCT/US2003/036617, filed Nov. 14, 2003, and claims the benefit of U.S. Provisional Patent Application No. 60/426,984 filed Nov. 15, 2002, both of which applications are incorporated herein by reference.

### FIELD

The present disclosure relates to surgical devices and methods, such as surgical devices and methods for the treatment of cardiac diseases and conditions. In particular, the methods for percutaneous or open-surgical treatment or repair of regurgitant cardiac valves.

### BACKGROUND

The four chambers of the mammalian heart pump blood throughout the body of an animal by rhythmically contracting in a regular pattern. In humans, the heart is divided into four chambers, including the left atrium and the right atrium (the upper cavities on each side of the heart) and the left ventricle and the right ventricle (the lower cavities on each side of the heart). Blood flows from the body through the venous system into two large veins, the superior vena cava and inferior vena cava that, along with the coronary sinus, empty into the right atrium. Contraction of the right ventricle forces blood from the right ventricle into the pulmonary artery and then to the lungs where it is oxygenated. Following contraction, blood flows from the right atrium into the right ventricle. A valve, named the tricuspid valve, separates the right atrium and right ventricle and prevents backflow of blood from the right ventricle into the right atrium during contraction. At the lungs, the pulmonary artery branches into a series of smaller arteries and capillaries where the blood is oxygenated. The oxygenated blood returns to the heart through a network of veins that empty into the four pulmonary veins, which connect to and route blood to the left atrium of the heart. Contraction of the left ventricle forces blood into the aorta and eventually into the network of arteries and capillaries that direct the flow of oxygenated blood back into the body. The left atrium and left ventricle are separated by the mitral valve, which, similar to the tricuspid valve, prevents backflow of blood into the left atrium when the left ventricle contracts. Following contraction of the left ventricle, blood flows from the left atrium into the left ventricle, where it is pumped through the aorta in the next contraction.

Regurgitation (leakage) of the mitral valve or tricuspid valve can result from many different causes, such as an ischemic heart disease, myocardial infarction, acquired or inherited cardiomyopathy, congenital defect, myxomatous degeneration of valve tissue over time, traumatic injury, infectious disease, or various forms of heart disease. Primary-heart-muscle disease can cause valvular regurgitation through dilation, resulting in an expansion of the valvular annulus and leading to the malcoaptation of the valve leaflets through overstretching, degeneration, or rupture of the papillary-muscle apparatus, or through dysfunction or malpositioning of the papillary muscles. This regurgitation can cause heart irregularities, such as an irregular heart rhythm, and itself can cause inexorable deterioration in heart-muscle function. Such deterioration can be associated with functional impairment, congestive heart failure and significant pain, suffering, lessening of the quality of life, or even death.

Surgical options for correcting defects in the heart valves include repair or replacement of a valve, but these surgical options require open-heart surgery, which generally requires stopping the heart and cardiopulmonary bypass. Recovery from open-heart surgery can be very lengthy and painful, or even debilitating, since open-heart surgery requires pulling apart the ribs to expose the heart in the chest cavity. Cardiopulmonary bypass itself is associated with comorbidity, including cognitive decline. Additionally, open-heart surgery carries the risk of death, stroke, infection, phrenic-nerve injury, chronic-pain syndrome, venous thromboembolism, and other complications. In fact, a number of patients suffering heart-valve defects cannot undergo surgical-valve treatment because they are too weak or physiologically vulnerable to risk the operation. A still larger proportion of patients have mitral-valve regurgitation that is significant, but not sufficiently so to warrant the morbidity and mortality risk of cardiac surgery. If there were a less dangerous—even if less effective—minimally invasive mechanical procedure, more patients would likely undergo mechanical treatment of valvular regurgitation.

Pharmacologic treatments for valvular regurgitation generally include diuretics and vasodilators. These medicines, however, have not been shown to alter the natural progression of cardiac dysfunction associated with regurgitant valves. Therefore, a need exists for treatment options that do not involve open-heart surgery or conventional medications.

### SUMMARY

Described herein are embodiments of a system and method for repair of cardiac valves, including (but not limited to) percutaneous and minimally invasive surgical procedures for the treatment of valvular regurgitation. The system and method involve transcatheter-mitral-valve-cerclage annuloplasty, transcatheter-leaflet reapposition (which can be considered a percutaneous Alfieri procedure), or a combination thereof.

An exemplary transcatheter-mitral-valve-cerclage annuloplasty involves the introduction of tensioning material around the mitral-valve annulus using a secondary catheter, such as a steerable guide wire or canalization catheter. Access to the area around the mitral-valve annulus can be accomplished using a number of different percutaneous approaches, including access from and through the coronary sinus. In particular embodiments, a continuous strand of tensioning material (for example, ligature) is applied around the mitral-valve annulus along a pathway that, in certain embodiments, includes an extraanotomic portion. For example (and without limitation), the tensioning material can traverse a region between the anterobasal-most portion of the coronary sinus and the coronary-sinus ostium. As another non-limiting example, tensioning material can be applied across the atrial aspect of the mitral valve from the posterolateral aspect to the anterior aspect of the coronary sinus, or from the septal aspect to the lateral aspect of the mitral-valve annulus. By cerclage, this procedure can reduce the mitral annular cross-sectional area, including a reduction in septal-lateral wall separation, thereby intrinsically reapposing the line of coaptation of the mitral valve.

An exemplary transcatheter-leaflet reapposition involves the percutaneous introduction of a suture-delivery device (for example, a device for delivering and applying a suture-clip-pledget assembly) to, for example, the anterior and posterior mitral-valve leaflets. Opposing clip-pledget assemblies, delivered onto a moving mitral leaflet on a beating heart, are susceptible to misalignment during delivery. However, in certain embodiments, even if the suture clips are applied in a misaligned or offset position, appropriate registration of the malaposed suture clips can be accomplished.

### BRIEF DESCRIPTION OF THE DRAWINGS

FIG. 1 is an anterior side view of a heart in partial cross-section illustrating an approach for introducing a guiding catheter and valve-manipulation catheter from the left atrium into the left ventricle of the heart.

FIG. 2 illustrates manipulation of a cardiac-valve leaflet by a valve-manipulation catheter. FIG. 2 is an enlarged, simplified view of the region of the heart engaged by the valve-manipulation catheter in FIG. 1, but with a guiding catheter approaching the valve from a different direction than the embodiment shown in FIG. 1. While FIG. 2 depicts a valve-manipulation catheter gripping the leaflet adjacent the corner of the leaflet, the leaflet can be gripped at any chosen portion of the leaflet, such as any portion of the free interior edge of the leaflet, including the middle of the leaflet's free edge.

FIG. 3 is an end elevation view of two cardiac-valve leaflets grasped by two suture clips, or staples, each one attached to the free edge of a leaflet.

FIG. 4 is a top view of another embodiment of the suture assembly illustrated in FIG. 3. In FIG. 4, two suture clips attached to leaflets of a cardiac valve are offset from each other, with the ligature segments of a suture extending therebetween prior to tensioning. Thus, tensioning of the ligature segments would urge the suture clips (and the valve leaflets) toward each other, leading to apposition of the valve leaflets. Different sutures can be chosen for tensioning after the suture-clip-pledget assembly is attached to the mitral valve, thereby permitting appropriate registration along the line-of-coaptation, as well as registration axially along the line of blood flow.

FIGS. 5A-5C illustrate the deployment of preformed secondary catheters from a guiding catheter, which may be delivered antegrade across the interatrial septum or delivered retrograde across the aortic valve.

FIGS. 6A-6B are top views of a cardiac valve illustrating two cerclage sutures following a transcatheter-cerclage annuloplasty. The suture may traverse the coronary sinus and mitral annulus exclusively (e.g., FIG. 6A) or may traverse in part the left or right atrial cavity (e.g., FIG. 6B).

FIG. 7 is a top view of a cardiac valve illustrating a transverse, continuous suture following a transcatheter annuloplasty. This form of cerclage has the effect of augmenting the line of mitral-valve coaptation by reapposing the septal and lateral aspects of the mitral annulus, and thereby reapposing the anterior and posterior mitral leaflets.

FIGS. 8A and 8B illustrate end and side views, respectively, of one embodiment of a guiding catheter.

FIGS. 9A-9B and 10A-10C show exemplary approaches for applying a cerclage suture to a mitral valve of a heart. FIGS. 9A-9B are top perspective views of a portion of the vasculature around the mitral valve showing the trajectory of the exemplary approaches. FIG. 9A shows one exemplary approach for applying a cerclage suture to the mitral valve. FIG. 9B shows an exemplary approach for a applying a transverse, continuous suture to the mitral valve. FIGS. 10A-10C are top perspective views illustrating the placement and advancement of a guiding catheter and a canalization catheter during the application of a cerclage suture along the trajectory shown in FIG. 9A. The sutures can bear tension-reduction devices (e.g., pledgets) to redistribute tension at sharp angles.

FIG. 11 is a top perspective view of a porcine heart with a cerclage suture along the trajectory shown in FIG. 9A.

### DETAILED DESCRIPTION

Recently developed imaging techniques, such as real-time magnetic resonance imaging (rtMRI), intracardiac, transesophageal, three-dimensional echocardiography, and electromagnetic three-dimensional guidance, can guide non-surgical heart valve repair using percutaneous-catheter techniques in awake patients. Because the risks and complications of surgery are reduced (compared with open-heart surgery), catheter-based heart-valve procedures are suitable for a broader array of patients. Disclosed herein are devices and methods for catheter-based valve repair that can be used to repair damaged or malfunctioning cardiac valves. Embodiments of the disclosed devices and methods can be used, for example, to re-appose valve leaflets by percutaneous-cerclage annuloplasty (reconstruction or augmentation of the ring or annulus of a defective cardiac valve) or to reappose malcoapted valves with appropriate leaflet registration. Included are devices and methods for delivering circumferential and radial tensioning devices by catheter-based annular cerclage and for catheter-based capture, alignment, and tensioning of valve leaflets.

These procedures can include using an imaging system to image the internal bodily tissues, organs, structures, cavities, and spaces of the subject being treated. For example, the systems and methods described herein can include transmitter or receiver coils to facilitate active-device navigation using an imaging system, such as magnetic-resonance imaging (MRI). This imaging can be conducted along arbitrary or predetermined planes using various imaging methods based on X-ray technologies, X-ray fluoroscopy, MRI, electromagnetic-positron navigation, video technologies (such as endoscopy, arthroscopy, and the like), ultrasound, and other such technologies. In some embodiments, real-time MRI (rtMRI), intracardiac ultrasound, or electromagnetic guidance is employed. Thus, as used herein, the term “imaging system” includes any device, apparatus, system, or method of imaging the internal regions of a subject's body.

The devices disclosed can include: a guiding catheter (GC), such as preformed guiding catheters designed to approach cardiac valves, such as the mitral valve, from a transaortic or a transseptal approach; an apparatus for capturing a valve leaflet and attaching a suture to the leaflet; a system for appropriate alignment of sutures, even if the suture clips or other suture anchors to a heart valve are misaligned; and a system for catheter-based delivery of an annuloplasty suture, such as a cerclage-annuloplasty suture, a circumferential-tensioning device, or a transverse suture across a heart valve. These devices and methods provide a new class of therapeutic-cardiac procedures that previously required open-heart or port-access heart surgery. The catheter-based treatments described herein can be applied to a wider range of patients, including patients not healthy enough for other forms of heart surgery, because these new treatments are less invasive.

The singular forms “a,” “an,” and “the” refer to one or more than one, unless the context clearly indicates otherwise. For example, the term “comprising a secondary catheter” includes single or plural secondary catheters and is considered equivalent to the phrase “comprising at least one secondary catheter.”

The term “or” refers to a single element of stated alternative elements or a combination of two or more elements. For example, the phrase “rtMRI or echocardiography” refers to rtMRI, echoradiography, or both rtMRI and echocardiography.

The term “comprises” means “includes without limitation.” Thus, “comprising a guiding catheter and a guide wire” means “including a guiding catheter and a guide wire,” without excluding additional elements.

The term “proximal” refers to a portion of an instrument closer to an operator, while “distal” refers to a portion of the instrument farther away from the operator.

The term “subject” refers to both human and other animal subjects. In certain embodiments, the subject is a human or other mammal, such as a primate, cat, dog, cow, horse, rodent, sheep, goat, or pig.

As used herein, the term “suture” is meant to encompass any suitable tensioning device and is not limited to only ligature-based sutures. It also includes tension-redistribution devices, such as pledgets, and instrinsic variations, such as altered diameter or stiffness.

As used herein, the term “guide wire” refers to a simple guide wire, a stiffened guide wire, or a steerable guide-wire catheter that is capable of puncturing and/or penetrating tissue.

Myocardial Catheter System

The system described herein can include several components: a guiding catheter (GC); a guide wire; a secondary catheter, such as a valve-manipulation catheter (VMC) or a canalization-needle catheter (CNC); and, in some embodiments, an implantable suture-clip-pledget (SCP) assembly or other tensioning device. In some embodiments, this system can be considered a myocardial-canalization system or other system for therapeutically treating the heart. This system is useful for repair or replacement of heart valves, for example, the mitral valve or tricuspid valves. The system can be used for other surgical procedures in addition to repairing or replacing cardiac valves, such as other minimally invasive surgical procedures.

The guiding catheter (GC) enables percutaneous access into a subject's body, for example, percutaneous access to the heart, such as a chamber of the heart. In some embodiments, the GC is designed for access to the left ventricle and/or the left atrium of the heart. The GC permits introduction of one or more secondary catheters, including a valve-manipulation catheter (VMC) or canalization-needle catheter (CNC) as described below. The secondary catheter (or catheters) is used to treat, affect, or manipulate an organ, tissue, or structure of interest in the subject's body, such as the heart or particular structures within the heart. If the GC is used for percutaneous (or other) access to the heart, the GC permits introduction of a secondary catheter, such as a VMC, into the heart while maintaining hemostasis.

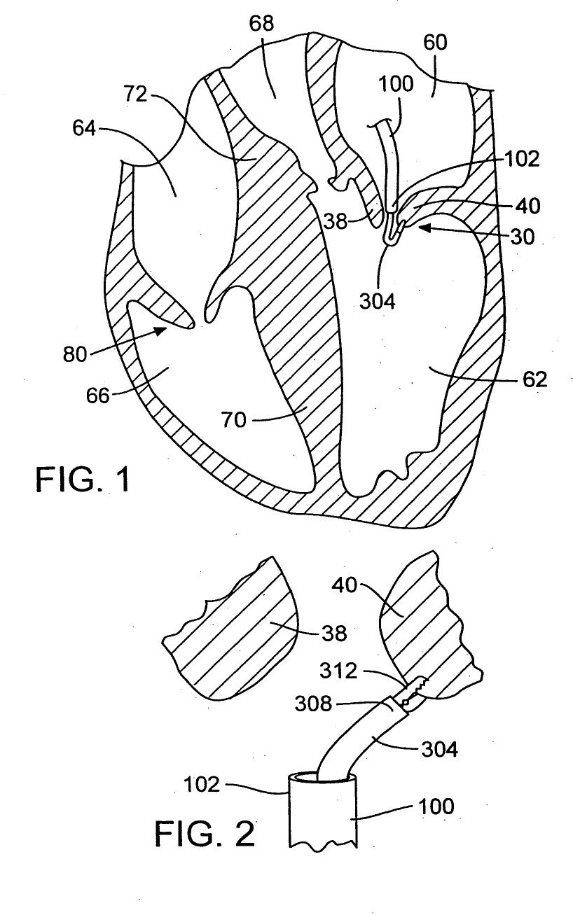

FIG. 1 illustrates one embodiment of the system viewed from the anterior side of a heart in partial cross-section through the left atrium 60, left ventricle 62, right atrium 64, right ventrical 66, aorta 68, ventricular septum 70, and atrial septum 72. Guiding catheter 100 is shown within the left atrium 60 with its distal end 102 adjacent the mitral valve 30. FIG. 2 is a closer view of GC 100 with a VMC 304 deployed from its distal end 102 and extending upwardly through the left ventricle, thus illustrating a different approach to the mitral valve than the approach illustrated in FIG. 1.

In FIG. 1, for sake of clarity in the drawing, GC 100 is shown entering the left ventricle 62 from the left atrium 60 via an approach (not shown) into the left atrium 60, and a substantial portion of the GC leading proximally away from the distal end 102 of the GC is not shown. Approaches that direct the GC into the left atrium are described herein. The illustrated approach is only one of the many approaches to the mitral valve (or other structure of the heart) described herein. For example, GC 100 could enter the left ventricle 62 via a transaortic approach, in which GC 100 would extend through the aorta 68, down into the left ventricle 62, then back up to approach the mitral valve 30 as shown in FIG. 2. As another example, GC 100 could be directed into the right atrium 64, via a transcaval approach, then into the left atrium 60 through the atrial septum 72 anterior to the aorta 68. Additionally, GC 100 could be directed from the right atrium 64 through the opening of the tricuspid valve 80, into the right ventricle 66, then through the ventricular septum 70 into the left ventricle 62. Each of these approaches (and the others described herein) is non-limiting in the sense that GC 100 can be directed into the heart via any suitable approach. The choice of approach to the heart can depend on various factors and considerations, such as (but not limited to) the type of repair or treatment to be conducted, the physiological condition of the heart, the overall physiological condition or health of the subject, and available methods or systems for imaging the subject's body.

GCs are available in different shapes to suit the appropriate component of the mitral-valve-repair procedure. For example, GC shapes can be provided to suit different coronary sinus with different radii of curvature, to suit transaortic as well as transseptal access routes, or to suit atria and ventricles of different calibers. All such shapes can be accommodated with appropriate primary, secondary, and tertiary curves.

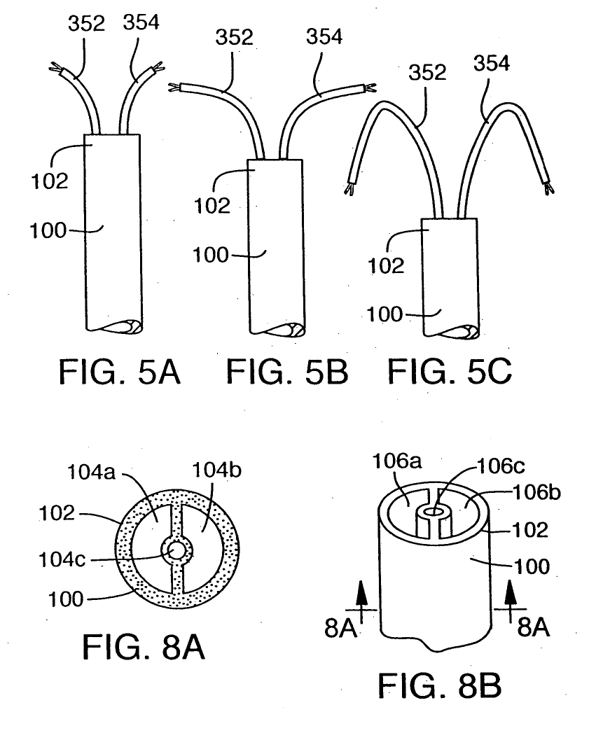

Different GCs are available to suit different tasks. For example, the GCs intended to guide cerclage annuloplasty can have different characteristics (such as, but not limited to, overall dimensions, lumen dimensions, shape, and steerability) compared with GCs intended to guide leaflet reapposition. The GC can be advanced and retracted to permit gross and/or fine axial positioning of the secondary catheter. The GC can also permit transmission of torque to reposition a secondary catheter adjacent a particular bodily structure, such as a particular valve of the heart. Additionally, the GC can be positioned axially relative to a preformed secondary catheter, such as one made from a shape-memory alloy, to alter the shape and deployment of a secondary catheter. For example (and without limitation), FIGS. 5A-5C illustrate the deployment of two preformed secondary catheters 352, 354 that retroflex as they emerge from the distal end 102 of the GC 100 during deployment. Thus, the secondary catheters 352, 354 can be straightened (by withdrawing them into the GC 100) during transvascular access and retroflexed for direct access to a valve leaflet during diastole. As shown in FIGS. 5A-5C, the retroflexed secondary catheters 352, 354 take on a configuration during or after deployment herein referred to as a “viper fang” or “ram's horn” configuration, due to their shape-memory feature. The secondary catheter shown in FIG. 1 is similarly deployed. Other preformed and shape-memory secondary catheters, however, can take on different shapes. The tension induced during retroflex of preformed secondary catheters can be used to manipulate tissues or structures of the subject's body. For example (and without limitation), FIG. 2 shows that a deployed VMC 304 has a clip 312 that can capture a portion of a valve leaflet (for example, posterior valve leaflet 40 in FIG. 2).

For percutaneous introduction of the GC, any appropriate percutaneous pathway and introduction method can be used, such as introducing the GC percutaneously into a blood vessel and then advancing it through the vasculature into a desired chamber of the heart. For example, the GC can be introduced percutaneously into a femoral artery by a cutdown of the artery or via a modified Seldinger technique, advanced through the femoral or brachial artery into the aorta, then through the aorta and across the aortic valve into the left ventricle. As yet another example, the GC can be introduced into a vein, such as the femoral or jugular vein, and guided through the inferior or superior vena cava into the right ventricle of the heart, or using a transseptal puncture, across the interatrial septum and into the left atrium and left ventricle. Moreover, a GC can access the coronary sinus from its ostium in the right atrium and from there around the mitral-valve annulus. However, the GC is not limited to percutaneous advancement into the heart (or even only selected chambers of the heart), but can be percutaneously introduced into other vascular or perivascular structures, such as the liver, the aorta, the lungs, stomach and intestines, colon and rectum, uterus, bladder, or even into a vascular or perivascular tumor. Thus, the descriptions of cardiac-valve repair included herein can be adapted for repair, treatment, or replacement of other cardiac structures (such as the interior myocardium), vascular structures, or perivascular structures. These transcatheter approaches do not require open-heart surgery and can be conducted in subjects who are awake and conscious (or semi-conscious) during the procedure. However, if necessary or desired, the system and uses described herein can be utilized and conducted during open-heart surgery, abdominal surgery, or the like, or in an anesthetized subject.

For purposes of this disclosure, percutaneous introductions of the GC into the heart can be classified into two (non-limiting) general approaches: an antegrade approach or a retrograde approach. The antegrade approach is conducted through the venous system, while the retrograde approach is conducted through the arterial system. As one, non-limiting example, an antegrade approach to the mitral valve of the heart involves introducing the GC into a vein (such as the femoral vein), advancing the GC through the inferior or superior vena cava into the right atrium, and then advancing the GC through a transseptal puncture into the left atrium and across to the mitral valve. As another non-limiting example, a retrograde approach to the mitral valve of the heart involves introducing the GC into an artery (such as the femoral artery) and guiding it into the aorta to the left ventricle. Additionally, in either approach, the GC can be extended through the vasculature and out of the body through another percutaneous opening. As just one non-limiting example, the antegrade approach described above can be extended by traversing the GC from the left atrium into the left ventricle, then into the aorta and out of the body through a second percutaneous opening in an artery, such as the femoral artery.

In addition to percutaneous introduction, the GC may be introduced into a target area or structure of the body via other methods. For example, the GC can be introduced via a transseptal puncture, a puncture through one of the intercostal spaces at a desired position, or some other standard-transcatheter approach. In fact, the system can be used in invasive surgeries, such as open-heart surgery, abdominal surgery, and the like, even though percutaneous surgical methods offer certain advantages over invasive surgeries (such as reduced risk of infection and shorter recovery time). Thus, the GC can be introduced via any suitable approach, including transaortic, transseptal-transmitral, and transcaval approaches.

Returning to FIGS. 8A and 8B, the GC 100 has a proximal end (not shown), a distal end 102, and a lumen 104. The GC 100 can be any suitable guidable or steerable catheter. In some embodiments (such as the embodiment illustrated in FIGS. 8A-8B), the GC lumen 104 is subdivided into separate lumens 104a, 104b, 104c, each of which is capable of holding a single secondary catheter or guide wire. In alternative embodiments, the GC lumen or subdivided parts of the GC lumen hold multiple secondary catheters, multiple guide wires, both a secondary catheter and a guide wire, or a combination of multiple secondary catheters and guide wires. One particular (and non-limiting) type of GC 100 is a guidable catheter having a guide-wire lumen 104c, such-as the GC illustrated in FIGS. 8A-8B. Thus, the guide-wire lumen 104c is one type of subdivided lumen. The guide-wire lumen 104c can be centrally located within the GC lumen, or it can be located in an offset position. When such a catheter is used, a guide wire (described below) is first inserted into the subject (percutaneously or non-percutaneously, as described above in relation to the GC) and advanced to the area of interest within the subject's body, such as a chamber of the subject's heart. The guide wire is slideably held within the guide wire lumen of the GC, and the GC is advanced along the guide wire into the body of the subject. For example, a guide-wire lumen in a GC can provide over-wire access into the left ventricle of a heart (for example, via a transaortic approach or transseptal approach) or into the left atrium of a heart (for example, via a transcaval or transseptal approach).

The dimensions of the GC can depend on several considerations, such as the physical characteristics and health of the subject treated and the methods and/or approaches used. In some embodiments, the GC is about 50 to 200 cm long and about 1 to 40 mm in diameter. In particular embodiments, the GC is about 80 to 100 cm long and about 1 to 3 mm in diameter. For example, a GC of about 130 to 150 cm in length with a diameter of about 3 mm can be introduced into the femoral artery in the groin of an adult human patient and guided into the left ventricle of the heart via a transaortic approach. Such a GC has pushability and movement characteristics comparable to contemporary 6 to 10 French diameter coronary-interventional catheters.

If a guide wire is used in conjunction with the GC, the guide wire is dimensioned to operate with the catheter and is usually longer than the GC. For example, a guide wire of about 100 to about 250 centimeters in length and about 0.1 to about 2 mm in diameter can be used with the GC described above. If a secondary catheter, such as a VMC, is intended for use with the GC, that secondary catheter also is dimensioned to operate with the GC and is usually longer than the GC. For example, a secondary catheter of about 100 to 250 cm long and about 1 to about 10 mm in diameter can be used with the GC described above.

While the GC described above is dimensioned for introduction into the femoral artery in the thigh of an adult human patient and guidance into the left ventricle of the heart through the aorta, devices for other uses, approaches, and/or for other subjects can be sized differently. For example, a device introduced into the brachial or radial artery of a human patient can be shorter in length, and a device used with a dog can have a shorter length and smaller diameter. Additionally, the GC, guide wire, and any secondary catheter (such as a VMC) can be any shape in cross-section, although some embodiments employ GCs, guide wires, and secondary catheters that are round, oval, or elliptical in cross-section.

The GC can be made of any suitable material or combination of materials that provide both the strength and flexibility suitable to resist collapse by external forces, such as forces imposed during bending or twisting. Exemplary materials include, but are not limited to: polymers, such as polyethylene or polyurethane; carbon fiber; or metals, such as Nitinol®, platinum, titanium, tantalum, tungsten, stainless steel, copper, gold, cobalt-chromium alloy, or nickel. The GC optionally can be composed of or reinforced with fibers of metal, carbon fiber, glass, fiberglass, a rigid polymer, or other high-strength material. In particular embodiments, the GC material is compatible with MRI, for example, braided Nitinol®, platinum, tungsten, gold, or carbon fiber. Additionally, the exterior surfaces of the GC can be coated with a material or substance, such as Teflon® or other lubricous material, that aids with the insertion of the GC into the body of the subject and/or aids in the movement of the GC through the subject's body.

The GC also can contain features that aid in imaging the position of the GC within the body of the subject, such as radioopaque markers or receiver coils to enhance visualization by fluoroscopy, MRI or X-ray, or etched grooves to enhance visualization by ultrasound imaging, including echocardiography. As another example, the GC can be coated with a T1-shortening or T2*-shortening agent to facilitate passive visualization using MRI. Additionally, the GC itself can contain its own visualization device, such as a fiber-optic cable having a lens at its distal end and connected to a video camera and a display unit at its proximal end. For example, the GC can contain a secondary catheter adapted from existing, commercially available endoscopes, such as various rhino-, naso-, pharyngo-, laryngoscopes and tracheal-intubation fiberscopes available from manufacturers such as Olympus®, Fujinon®, Machida®, and Pentax®.

The GC can be connected to any appropriate surgical apparatus, such as a syringe, infusion pump, or injection catheter that can pump a solid, liquid, or gaseous substance into a lumen of the GC. As one specific non-limiting example, the GC can include a syringe containing sterile saline solution in fluid connection with the GC lumen. The operator of the device can use the syringe to flush an area adjacent the distal end of the GC by injecting the saline solution into the GC lumen and pressurizing the lumen, thereby forcing the saline solution out through the distal lumen port. U.S. Pat. No. 6,346,099 provides one non-limiting example of an injection catheter. As another non-limiting example, the GC can be operably coupled to a hemostatic y-adaptor, such as a Tuohy-Borst side-arm adaptor.

The GC can be multi-catheter compatible, meaning that one or more secondary catheters, such as a valve-manipulation catheter (VMC), can be inserted into and through the GC lumen. In some embodiments, the internal portion of the GC is subdivided into multiple lumens, such as a guide-wire lumen and plural secondary-catheter lumens A GC lumen (including a guide-wire lumen or secondary-catheter lumen) can extend to a distal lumen port defined in a portion of the GC wall adjacent or at the distal end of the GC. Such lumen ports, including a guide-wire lumen port 106c and VMC-lumen ports 106a, 106b are illustrated in FIG. 8B.

Additionally, the GC can include a deflectable tip, such as a simple deflectable tip having a single degree of axial freedom. Exemplary (non-limiting) fixed-fulcrum and moveable-fulcrum-deflectable-tip catheters are commercially available, such as the deflectable-tip catheters described in U.S. Pat. Nos. 5,397,321; 5,487,757; 5,944,689; 5,928,191; 6,074,351; 6,198,974; and 6,346,099. Thus, any suitable fixed-fulcrum or moveable-fulcrum deflectable-tip catheter can be adapted for use as a GC disclosed herein. The GC also can include structures or mechanisms for aiding in the rotation of the catheter about its longitudinal axis.

The GC can include a guide collar, handgrip, handle, and other structures or devices at its proximal end (not shown) that aid in operation of the GC. Various control mechanisms, including electrical, optical, or mechanical control mechanisms, can be attached to the catheter via a guide collar (not shown). For example, a guide wire can be included as a mechanical control mechanism. The guide collar can include additional operational features, such as a grip for aiding manual control of the GC, markers indicating the orientation of the GC lumen or subdivided lumens, markers to gauge the depth of GC advancement, instruments to measure GC operation or physiological signs of the subject (for example, a temperature gauge or pressure monitor), or an injector control mechanism coupled to the GC lumen for delivering a small, precise volume of injectate. In some embodiments, the guide collar contains instrumentation electrically coupled to metallic braiding within the GC, thus allowing the GC to simultaneously be used as a receiver coil for MRI.

A guide wire used with the system for guiding the GC into and through a subject's body can be composed of any suitable material, or combination of materials, including the materials described above in relation to the GC. Exemplary (non-limiting) guide wires are composed of material having the strength and flexibility suitable for use with the device, such as a strand of metal (for example, surgical stainless steel, Nitinol®, platinum, titanium, tungsten, copper, or nickel), carbon fiber, or a polymer, such as braided nylon. Particular (non-limiting) guide wires are composed of a strand of Nitinol® or other flexible, kink-resistant material.

Similar to the GC, the guide wire can include an image-enhancing feature, structure, material, or apparatus, such as a radiopaque marker (for example, a platinum or tantalum band around the circumference of the guide wire) adjacent its distal end. As another example, the guide wire can include plural etchings or notches, or the guide wire can be coated with a sonoreflective material to enhance images obtained via intravascular, intracardiac, transesophogeal, or other ultrasound-imaging method. As another example, the guide wire can be coated with a T1-shortening or T2*-shortening agent to facilitate passive visualization using MRI. As yet another example, a fiber-optic secondary catheter can be inserted into and through a secondary-catheter lumen of the GC to assist in visualizing the position of the guide wire within the subject as a guide wire is deployed through the distal guide-wire lumen port.

Additionally, as similarly described in relation to the GC, the guide wire can contain a layer or coating of a substance, compound, or material that facilitates guide-wire insertion into and movement through the body of a subject, for example Teflon® or other hydrophilic or lubricous material.

In some embodiments, the guide wire and/or GC includes a structure, apparatus, or device at its distal tip useful for penetrating tissue, such as myocardial skeleton, muscle, or connective tissue. For example, the distal tip of the guide wire can be sharpened to a point for puncturing through tissue, or a secondary catheter having a coring mechanism or forceps at its distal tip can be used in conjunction with the GC. However, in alternative embodiments, the distal end of the guide wire is bent to provide a J-shaped or a pigtail-shaped tip to protect against perforation of tissue by the guide wire during manipulation. In still other alternative embodiments, the guide wire itself has a deflectable tip to facilitate traversal of tissue irrespective of natural tissue planes.

If a guide wire is used to guide the GC, the guide wire can be removed at any time after insertion of the GC into the body of the subject. For example (and without limitation), the guide wire can be removed after the distal end of the GC has traversed to about the same location as the distal end of the guide wire. Alternatively, the guide wire can be left in place inside the guide-wire lumen of the GC, in which case it can act as a receiver coil or antenna for certain imaging methods, such as MRI. Thus, the guide wire can serve to enhance the imaging of the GC following introduction of the GC into the body of the subject.

One or more secondary catheters can be deployed within the lumen of the GC. Like the GC, each secondary catheter has a proximal end and a distal end; however, not all secondary catheters have a lumen. For example, non-lumen secondary catheters can include various probes, such as temperature probes, radiofrequency or cryogenic ablation probes, or solid needles. An exemplary non-limiting secondary catheter is a valve-manipulation catheter (VMC), which can be deployed through the GC and into a chamber of the heart in order to contact and manipulate various cardiac valves.

As illustrated in FIG. 2, the distal end 308 of the VMC 304 can include a device 312 to capture a valve leaflet. The illustrated capture device is a spring-loaded clipping mechanism under the control of the system operator, similar to an alligator clip, but the VMC can have alternative devices, such as a device similar to the tips of a set of straight or curved forceps (for example, tissue forceps or alligator forceps), the tips of a straight or curved hemostat, or similar to the tip of a retractor (for example, a Senn-Mueller retractor). Other alternative capture devices include one or more bent probes or tongs, or one or more straight or curved needle tips. Thus, these devices can be considered means for capturing a valve leaflet.

In some embodiments, the VMC includes a bifurcated end with two tips of the same length or different lengths. For example (and without limitation), a VMC can include a long spatulated tip to appose to one surface of a targeted valve leaflet (such as the ventricular surface of a mitral valve leaflet) and a shortened spatulated tip to appose to another surface of the targeted valve leaflet (such as the atrial surface of a mitral valve leaflet). Such a spatulated tip permits the VMC to be pressed against the leaflet to capture it during movement, such as capturing a mitral-valve leaflet during diastolic opening. Additionally, the tension exerted by a VMC (transmitted, for example, by retraction of a retroflexed VMC) can manipulate the captured valve leaflet, such as pushing or pulling the mitral-valve leaflet toward a closed position.

A VMC also can include a closure mechanism, such as a mechanism analogous to biopsy forceps or a spring-operated clip (such as illustrated in FIG. 2), for capturing a bodily tissue or structure, such as a cardiac-valve leaflet. For example (and without limitation), such a closure mechanism can be employed to appose the spatulated tips described above.

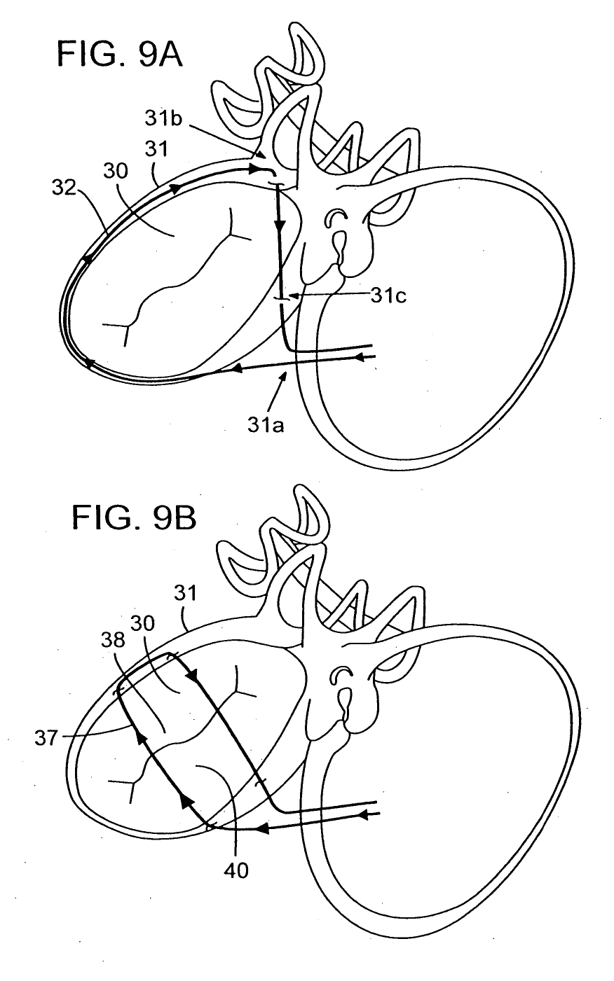

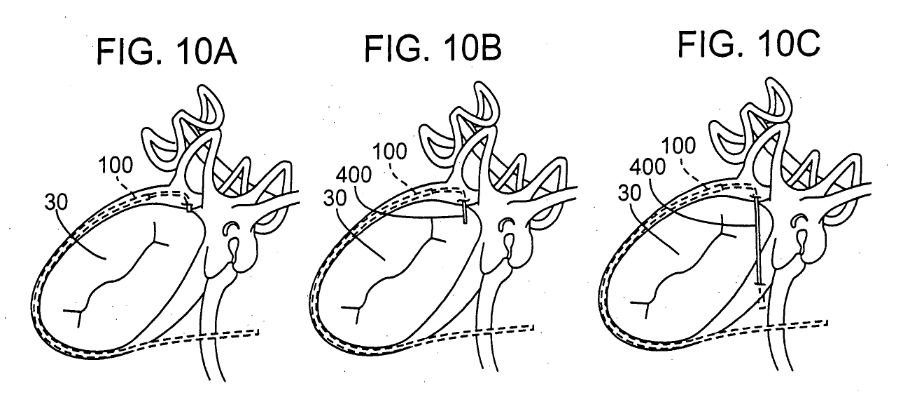

A canalization-needle catheter (CNC) is a type of secondary catheter that can be used to apply a suture to a bodily tissue, organ, or structure of interest. For example, as illustrated in FIGS. 9A-9B and 10A-10C, a GC 100 can be used to guide a CNC 400 to the mitral valve. The CNC 400 can be used to apply a circumferential suture, such as a cerclage suture, around the valve. This exemplary procedure is described in further detail below. CNCs can be adapted from existing canalization- or recanalization-needle catheters, such as those described in WO 94/13211 and U.S. Pat. No. 6,423,080

Similar to a GC, a secondary catheter can include a guide collar and other structures or devices at its proximal end that facilitate its operation. The control mechanisms, instrumentation, and other devices described above in relation to a GC also can be used with a secondary catheter. Moreover, the structures, apparatus, and devices described above in relation to a GC and used for penetrating tissue at the distal end of the GC also can be implemented in a secondary catheter.

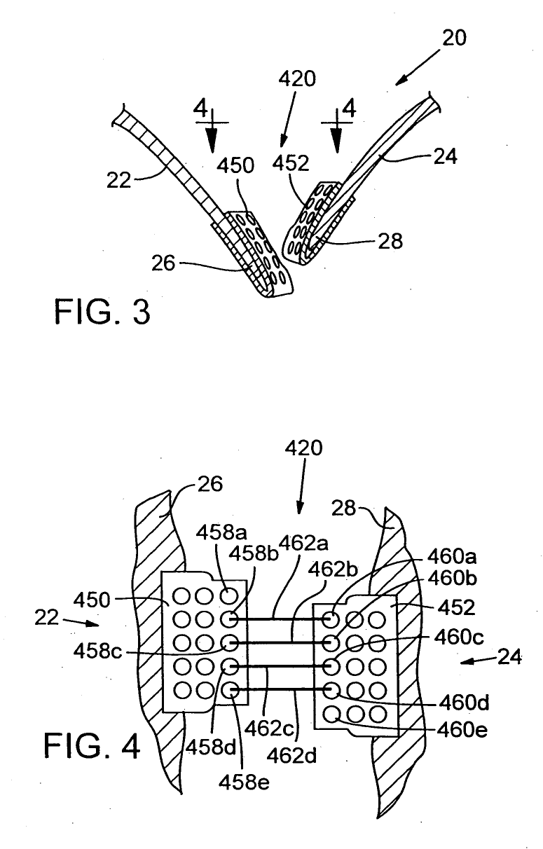

An implantable suture-clip-pledget assembly (SCP) is an implantable staple assembly for anchoring multiple adjacent interrupted pledget sutures to a tissue, structure, or organ of interest, for example (and without limitation), a valve-leaflet edge. The SCP can be designed for implantation on a permanent, semi-permanent, or temporary basis, although some embodiments employ a permanently implantable SCP. An SCP can have a low profile to reduce or minimize interference with the function of a target tissue, organ, or structure. For example, FIGS. 3 and 4 show two low-profile suture clips 450, 452 comprising an SCP 420 that reduces or minimizes interference of the SCP with blood flow through a valve.

The suture clip contains a mechanism for attachment to a tissue, organ or structure of interest, such as an anchor, grip, staple, or locking mechanism. For example, FIGS. 3 and 4 show alternative embodiments of two suture clips 450, 452, each with a gripping mechanism that captures respective portions of free edges 26, 28 of the valve leaflets 22, 24 and locks the suture clips into place on the respective valve leaflets. Additionally, a suture clip includes a structure or anchor point for attachment of a ligature, such as a hole bored through the suture clip or a hollow ring mounted on the surface of the suture clip. Multiple ligature anchor points can be included on a suture clip. For example, FIG. 4 illustrates suture clips 450, 452 with multiple bored holes, some of which are referenced by numbers 458a-e and 460a-e. It will be seen in FIGS. 3 and 4 that the suture clips 450, 452 have multiple rows of bores in selected orientations to permit placement of ligatures for producing desired effects during tensioning, such as relative movement of cardiac valve leaflets toward each other for reapposition. For the sake of clarity in the drawings, only some, but not all, of the bored holes are indicated with reference numbers.

An SCP can have a larger cross-sectional area than the suture alone. This feature can provide some advantage, depending on the use of the SCP. For example, an SCP with a larger cross-sectional area than the suture alone that is attached to a valve leaflet can buttress the valve leaflet against tension transmitted through the suture. An SCP can be delivered by a secondary catheter, such as a VMC, to the site of interest. For example, the distal end of the GC can be placed adjacent a cardiac-valve leaflet, and a secondary catheter carrying an SCP at its distal end (for example, a VMC) can be inserted through the GC and deployed through the distal end of the GC. Once deployed, the operator can manipulate the GC or the secondary catheter into a position where the SCP can be attached to the valve leaflet.

Multiple suture clips can be deployed to a single tissue, organ, or structure in the subject's body, or to adjacent tissues, organs, or structures. For example, as shown in FIG. 4, a first suture clip 450 and a second suture clip 452 are deployed opposed to each other on the edges 26, 28 of two valve leaflets 22, 24. In some cases, sutures between plural suture clips: require proper alignment in order to optimize the physiological benefits of putting such sutures in place. For example, proper alignment of sutures between two malcoapted cardiac leaflets can be necessary in order to reduce or eliminate regurgitation through the valve. However, it can sometimes be difficult for an operator to properly align multiple suture clips in some applications. For example, placing two suture clips in exact alignment on the separate leaflets of a moving cardiac valve, such as on a mitral valve while the subject's heart is beating, can be quite difficult. Therefore, an SCP can include a feature or mechanism that allows alignment of sutures between or among multiple suture clips even when the suture clips themselves are out of alignment. Moreover, the reapposition of the valve leaflets can be accomplished in the axial and/or radial dimensions.

For example, FIG. 4 shows two suture clips 450, 452 mounted on the edges 26, 28 of the two leaflets 22, 24 of a cardiac valve of the heart. The two suture clips are not in alignment, since the second suture clip 452 is offset from the first suture clip 450 (i.e., the second suture clip is shifted in the “downward” direction in FIG. 4). Each suture clip includes a series of regularly spaced-apart holes (such as the holes numbered 458a-e, 460a-e, and the other non-numbered holes) that can receive ligatures. If a ligature is connected to the first hole 458a of the first suture clip 450 and the first hole 460a of the second suture clip 452, then the tension in the resulting suture could aggravate the condition of the valve leaflets. However, as shown in FIG. 4, the sutures can be properly aligned by passing ligatures 462a-d through particular holes of each suture clip. In FIG. 4, for example, the second hole 458b of the first suture clip 450 is connected by a ligature segment 462a to the first hole 460a of the second suture clip 452, the third hole 458c of the first suture clip 450 is connected by a ligature segment 462b to the second hole 460b of the second suture clip 452, and so on. Thus, the suture is properly aligned to reappose the cardiac valve leaflets 22, 24. Consequently, the regurgitation through the valve can be reduced or eliminated, even though the suture clips were placed in misaligned positions. Suture clip alignment along other axes can be accomplished in different directions by passing ligature segments through different holes of suture clips 450, 452, as shown in FIGS. 3 and 4. In fact, suture clips with particular arrangements of ligature anchor points (such as the illustrated holes) can be pre-selected according to the direction(s) of realignment required to reappose the cardiac valve leaflets.

Ligatures used for the various sutures described herein can be composed of any suitable material, such as surgical cotton, cotton tape, linen, or other natural fiber; nylon, polyester, or other polymer; metal, such as surgical stainless steel; carbon fiber; or surgical gut. In some embodiments, however, surgical staples composed of the same or similar materials can be used in place of ligatures. Ligature materials can be used in a woven, braided, or monofilament form. Suitable ligature and suture materials are commercially available from Ethicon, Inc. (Somerville, N.J.) and other companies.

### Exemplary Embodiments

The following descriptions relate to exemplary embodiments for repairing the mitral valve of the heart.

Percutaneous-Transmyocardial-Cerclage Annuloplasty Using Tension Sutures

This embodiment is directed at (but not limited to) treating Carpentier-Type-I mitral-valve regurgitation, in which valvular regurgitation is related to annular dilation associated with underlying cardiomyopathy. In the Carpentier-Type-I condition, valve-leaflet mobility and alignment are normal, but the leaflets do not sufficiently appose one another to prevent regurgitation of blood into the left atrium. This lack of valvular apposition can result from a variety of diseases or physiological defects, such as myocardial-annular dilation following a myocardial infarction or non-ischemic cardiomyopathy. While this description relates to the mitral valve, this procedure can be readily adapted to other cardiac valves, such as the tricuspid valve, or other similar tissues and structures of a subject's body.

Briefly, a guiding catheter is inserted percutaneously into the vasculature of a subject, such as into the femoral vein, and guided through the vasculature into the heart. Access to the mitral valve can be accomplished in a variety of ways, such as a jugular or femoral transvenous approach to the coronary sinus through the right atrium, a transaortic approach into the left ventricle, a transseptal approach into the left atrium, or in any other suitable manner. Additionally, a non-percutaneous approach can be employed, if necessary or desired. Once the distal end of the GC is in place, a canalization needle catheter (CNC) is introduced into the lumen of the GC and traversed through the GC. According to one exemplary embodiment, the distal end of the CNC is advanced and directed under imaging guidance around the circumference of the cardiac valve. The advancement of the CNC can be performed in coordination with the GC in order to further advance the GC or related catheter into a circumferential position to permit capture and delivery of a circumferential-suture device. One exemplary circumferential trajectory of the CNC-GC apparatus is around the mitral-valve annulus from the coronary sinus ostium to the origin of the great cardiac vein, and thereafter through non-anatomic spaces (including but not limited to, the mitral annulus, left atrial cavity, right atrial cavity, interatrial septum, and transverse fossa) to return to the coronary sinus ostium. By virtue of anatomic variation, should the mitral-valve annulus not be in plane with the coronary sinus, alternative non-anatomic trajectories can be followed.

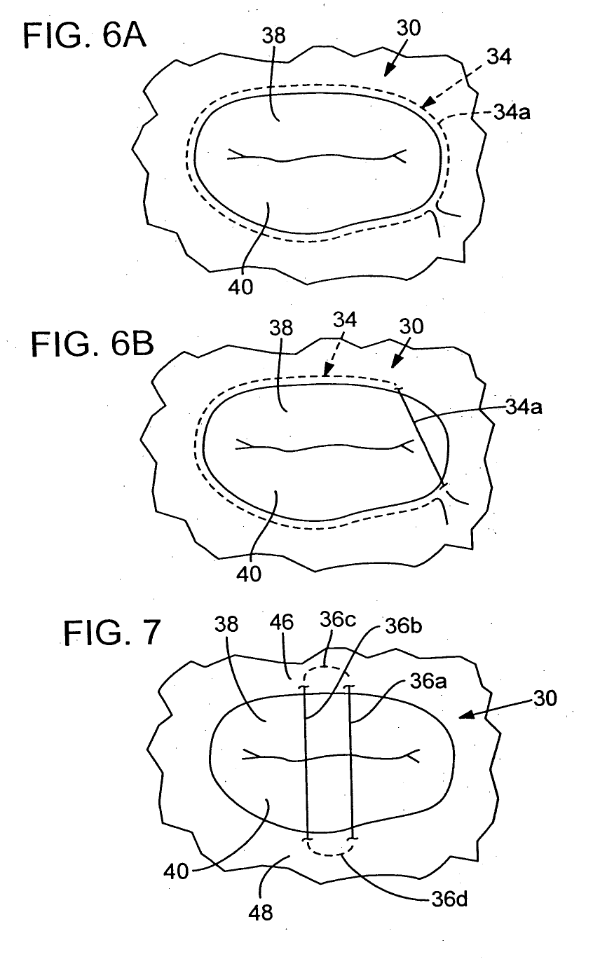

The type of suture applied to the valve can vary according to factors or considerations, such as the needs or desires of the surgeon, the nature of the valve defect, or the availability of equipment or supplies. In some embodiments, the suture is a cerclage or other type of circumferential suture (as illustrated in FIGS. 6A-6B) or a transverse suture (as illustrated in FIG. 7). The suture also can be a combination of different types of sutures, such as a partial or complete cerclage and a partial or complete transverse suture.

A suture can be applied using any suitable device, apparatus or method. Exemplary devices, apparatus, and methods include (but are not limited to) those described in U.S. Pat. Nos. 5,860,992; 5,571,215; 6,033,419; 5,452,733; and WO 97/27799, and the references cited therein.

As illustrated in FIGS. 6A-6B, 9A, and 10A-10C, the circumferential cerclage-suture approach is based on an intravascular/intramuscular annuloplasty performed using tension sutures. These tension sutures can be introduced in a variety of ways, such as those described above. In particular embodiments, a suture is introduced by a device, such as a CNC, that traverses at least partially through the coronary sinus via the coronary-sinus ostium. The suture is then placed around the mitral annulus, and the CNC (or other device) is withdrawn back through the coronary-sinus ostium (see FIGS. 6A-6B, 9A, and 10A-10C). FIG. 6A illustrates schematically a cerclage suture 34 around the anterior leaflet 38 and posterior leaflet 40 of the mitral valve 30 of a subject prior to tying off or anchoring the ligature ends. In FIG. 6A, the suture 34 includes a transverse-ligature portion 34a that extends through a wall of the coronary sinus and through tissue space between the great cardiac vein and the coronary-sinus ostium. FIG. 6B illustrates schematically an alternative trajectory of the cerclage suture 34 that includes a transverse-ligature portion 34a which is more exposed than in FIG. 6A. The transverse-ligature portion 34a in FIG. 6B extends through a wall of the coronary sinus and traverses an exposed region adjacent the atrial aspect of the mitral valve 30 to a region near the coronary-sinus ostium.

FIG. 9A is another illustration schematically showing the circumferential trajectory 32 from FIG. 6B. FIG. 9A shows a portion of the vasculature around the mitral valve 30 and the tricuspid valve (not shown), including the coronary sinus 31 as it extends around the mitral-valve annulus. The illustrated trajectory 32 extends from the coronary-sinus ostium (shown generally as region 31a), through the coronary sinus 31, to a region 31b adjacent the great cardiac vein. Region 31b can also be established or referenced as the anterobasal-most portion of the coronary sinus 31 or the distal portion of the coronary sinus. From region 31b, the trajectory 32 traverses the atrial aspect of the mitral valve 30 and reenters the coronary sinus 31 at a region 31c near the coronary-sinus ostium 31a (for example, near the base of the intraventricular septum). As was shown in FIGS. 6A and 6B, the transverse-ligature portion between region 31b and 31c may be established through interposed tissue or through an exposed space in the left atrium of the subject.

The tension suture (such as a circumferential or cerclage suture) can be introduced by image-guided traversal of interposed tissue using a steerable or deflectable-tip transmyocardial canalization needle. For example, as illustrated in FIGS. 10A-C, the canalization needle 400 can be extended from the distal end of the GC 100 and directed to traverse the myocardial base from the distal coronary sinus to the base of the intraventricular septum, where it, reenters near the origin of the coronary sinus.

Once the positioning ligature is inserted, tension can be introduced into the suture by manipulating the ligature threads (for example, using another secondary catheter, such as a tension catheter that captures and anchors an end of the ligature). As tension is applied, valvular regurgitation of the mitral valve 30 is assessed repeatedly and non-invasively. After the valvular regurgitation has been reduced (or even eliminated) and a desired tension is achieved, the tension is fixed using a knot-delivery system (for example, from a knot-delivery catheter). If the resulting circumferential suture is knotted to form a closed loop, the suture essentially becomes a cerclage suture. Tension in the suture can also be released (for example, using another secondary catheter, such as a catheter with a suture-release blade) in order to readjust or remove the tension suture.

In alternative embodiments, direct pledgeted or tension sutures are implanted within the bases 46, 48 of the anterior 38 and posterior 40 mitral-valve leaflets. For example, FIG. 7 shows two transverse-suture portions 36a, 36b extending across the atrial aspect of the mitral valve 30 and connected by radial-suture portions 36c, 36d (indicated by dashed lines) to form a continuous suture.

FIG. 9B is another illustration schematically showing a trajectory 37 similar to the trajectory for the suture shown in FIG. 7. In FIG. 9B, the illustrated trajectory 37 extends through the coronary-sinus ostium into the coronary sinus, where it traverses from the posterolateral aspect to the anterior aspect of the mitral-valve annulus and back. The resulting suture supplies tension sufficient to reappose the anterior mitral valve leaflet 38 and the posterior mitral valve leaflet 40 without substantially interfering with the opening or closing of the mitral valve during its movement. In another example, suture clips or tension sutures can be implanted on the atrial surface of the mitral valve and connected to the bases of the anterior and posterior mitral valve leaflets. The disclosed trajectories should not be construed as limiting in any way, as there exist other possible trajectories, which may involve one or more transverse-ligature portions. For example, one or more radial sutures can be applied across the atrial aspect of the mitral valve from the septal to the lateral aspect of the mitral valve.

In either type of suturing (circumferential or radial), left atrial access to the mitral valve can be gained using a transseptal puncture, in addition to or in place of access through the coronary-sinus ostium or other access point. Thus, mitral-valve access can be accomplished through (but is not limited solely to) coronary-sinus access and trans-coronary-sinus access or puncture. Additionally, image guidance can employ rtMRI or sonography in a short-axis view visualizing the mitral-valve annulus and employing multiple interleaved planes of visualization, such as several planes parallel to the annular plane of the mitral valve and an orthogonal plane showing a catheter en face.

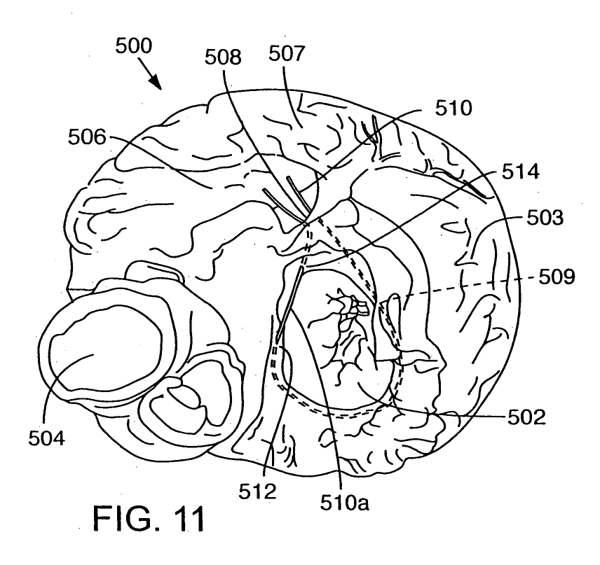

Experiments have been performed verifying the viability of the cerclage-suture trajectory 32 illustrated in FIG. 9A. In particular, and with reference to FIG. 11, a cerclage suture 510 was inserted into an explanted porcine heart 500 using the trajectory shown in FIG. 9A. FIG. 11 is a perspective view of the porcine heart 500 with the left and right atriums unroofed looking toward an atrial surface 502 of the mitral valve. By way of reference, FIG. 11 also shows the left ventricle 503, the aorta 504, the right atrium 506, the right ventricle 507, and the coronary-sinus ostium 508. The cerclage suture 510 comprised a nylon 2-0 suture, which was inserted into the coronary-sinus ostium 508, around the mitral-valve annulus through the coronary sinus 509, to an exit point 512, where the suture extended through the vasculature wall of the coronary sinus. The exit point 512 is generally positioned near the anterobasal-most portion of the coronary sinus, at or near the junction with the great cardiac vein. From the exit point 512, the suture 510 traversed a region of the left atrium to a reentry point 514, thereby forming a transverse-ligature portion 510a of the suture 510. At the reentry point 514, the suture 510 reentered the coronary sinus 509 near the coronary-sinus ostium 508. The nylon suture was replaced by cotton tape pulled through the circumferential trajectory. Once in position, the ends of the resulting cerclage suture were tensioned to reappose the mitral-valve leaflets.

In other experiments, alternative trajectories have been established and tested as viable cerclage-suture pathways. For example, in one experiment, a cerclage suture around the mitral-valve annulus was established by entering the coronary sinus through the superior vena cava, traversing along the coronary sinus to the coronary-sinus apex, crossing the fossa ovalis from the right atrium into the left atrium, and reentering the coronary sinus to complete the cerclage.

Percutaneous-Valve-Leaflet Reapposition

This embodiment is directed at (but not limited to) Carpentier-Type-II defects of the mitral valve, in which there is excessive leaflet mobility within the valve leading to malcoaptation of the mitral-valve leaflets. Causes of Carpentier-Type-II defects include degeneration or elongation of the valve leaflets, chordae, or papillary muscles. This degeneration can be myxsomatous or have some other degenerative effect or condition. Additionally, ischemic, infective, or traumatic injury to the mitral valve apparatus can cause Carpentier-Type-II defects. While this description relates to the mitral valve, this procedure can be readily adapted to other cardiac valves, such as the tricuspid valve, or other similar tissues and structures of a subject's body.

Briefly, one or more suture clips are applied to each leaflet of a cardiac valve via an approach beginning with the percutaneous insertion of a GC into the vasculature of a subject, such as the femoral artery of the subject. The operator, assisted by an imaging system, traverses the distal end of the GC through the vasculature and positions the distal end of the GC adjacent the mitral valve. Access to the mitral valve can be accomplished in a variety of ways, such as a transaortic approach into the left ventricle, a transseptal approach into the left atrium, or in any other suitable manner. Additionally, a non-percutaneous approach can be employed, if necessary or desired.

Once the GC is in place, a VMC is directed through the lumen of the GC to position the distal end of the VMC adjacent the mitral valve in sufficient proximity to capture a leaflet of the mitral valve. It is often not necessary to direct only one VMC (or only a single secondary catheter) through the GC at a time; multiple VMCs and/or secondary catheters can traverse through the GC lumen at the same time. After the distal end of the VMC is deployed from the GC, the operator uses the VMC to capture a portion of a leaflet of the mitral valve, such as capturing the leaflet along its free edge. A suture clip is then coupled to the leaflet of the cardiac valve using the same VMC or a different secondary catheter. A second suture clip is applied to a different leaflet of the cardiac valve in a similar manner. For example, the operator can use a secondary catheter to couple a suture clip to the anterior mitral-valve leaflet, withdraw the secondary catheter from the GC, reload the same secondary catheter with another suture clip, then direct the secondary catheter through the GC to couple a second suture clip to the posterior mitral-valve leaflet. As another example, the operator can direct two secondary catheters through the GC, couple a suture clip to the anterior valve leaflet with the first secondary catheter, then couple a second suture clip to the posterior valve leaflet using the second secondary catheter. After the suture clips are in place, one or more ligatures are run between the suture clips and tension is applied to the suture (and, thus, the suture clips) to realign the valve leaflets. While the intended result of this procedure is properly coapted valve leaflets, it is not necessary to achieve precise positioning and coaption of the leaflets leading to complete elimination of regurgitation through the valve. In fact, in some cases, perfect coaption is not possible for a variety of reasons, such as the physiological condition of the subject or potential interference between (or among) the suture clips and ligature segments. However, any significant realignment of the valves can reduce regurgitation and improve the subject's physiological condition.

Reapposing malcoapted valve leaflets to substantially fit together again can depend on proper alignment of the sutures between the suture clips. However, substantial (or even considerable) alignment of the sutures can be accomplished even when the suture clips are substantially offset from each other, as illustrated in FIGS. 3 and 4. As shown in FIGS. 3 and 4, the mitral-valve leaflets can be reapposed using a suture composed of several ligature segments 462a-d that induce tension directed towards the center of the valve. Sutures other than the illustrated suture, such as a figure-eight suture, also can be employed. Even though the suture clips are offset from one another (i.e., not in perfect opposition to each other), the suture is substantially aligned between the two leaflets. As illustrated, this is accomplished by running ligature segments between the substantially opposed holes of the suture clips and inducing tension in the ligature segments to draw the leaflets together. Thus, this reapposition can be considered a percutaneous delivery of an “Alfieri”-type surgical repair in which leaflets are reapposed using a figure-eight suture towards the center of the leaflets. See, e.g., Maisano et al., “The Edge-to-Edge Technique: A Simplified Method to Correct Mitral Insufficiency,” Eur. J. Cardiothorac. Surg. 13:240-6 (1998).

In some embodiments, the valve-manipulation catheter (VMC), or other secondary catheter, has a shape-memory characteristic, induced by a polymer or Nitinol®, that causes the secondary catheter to assume a preformed shape once it is released from the outer guiding catheter (GC), as illustrated in FIG. 2 and FIGS. 5A-5C. The VMC can take on any suitable preformed shape or curvature, depending on such factors as the size and condition of the organ, tissue, or structure to be manipulated. For example, the shape or curvature of the VMC can depend on the size of the heart or cardiac chamber, the shape of the heart valve, or the percutaneous approach to be used in deploying the system, such as an approach through the vasculature in a transseptal or transaortic approach to the mitral valve, or an IVC or SVC approach to the tricuspid valve.

The VMC also includes a grasping mechanism at its distal end, such as a clip, hook, clamp, or other mechanism capable of grasping a valve leaflet. Such catheters facilitate remote access to the free edges of the leaflet. Multiple VMCs can be deployed within a single guiding catheter (or multiple guiding catheters) to capture the free edges of multiple valve leaflets, and two or more VMCs can be deployed to capture the free edge of a single valve leaflet in multiple positions along that edge.

Once the free edge of a valve leaflet is captured by a VMC, a suture clip or clamp is attached (for example, by the VMC) for adjustable-reapposition, as shown in FIGS. 3 and 4. In certain embodiments, each suture clip has one or more pre-implanted sutures that can be selected to re-register and re-appose the leaflet edges together. Once the appropriate suture pairs are identified, tension is delivered percutaneously (as described above) and the efficacy of this repair can be tested by noninvasive assessment of valvular regurgitation. After the repair is made, the suture tension is secured permanently with knots and the unused, remaining sutures are ligated and removed.

Having illustrated and described the principles of the invention by several embodiments, it should be apparent that those embodiments can be modified in arrangement and detail without departing from the principles of the invention. Thus, the invention includes all such embodiments and variations thereof, and their equivalents.

1. A method for applying a suture to a cardiac valve, comprising:
percutaneously inserting a guiding catheter into he vasculature of a subject, wherein the guiding catheter has a proximal end, a distal end, and a catheter lumen;
traversing the distal end of the guiding catheter through the vasculature to position the distal end of the guiding catheter in a first position adjacent the cardiac valve;
traversing a canalization-needle catheter through the catheter lumen, wherein the canalization-needle catheter has a proximal end and a distal end;
positioning the distal end of the canalization-needle catheter in second position adjacent the cardiac valve and in sufficient proximity to apply a suture to the cardiac valve; and
applying a suture to the cardiac valve.

2. The method according to claim 1, wherein the positioning of the distal end of the canalization-needle catheter includes puncturing the vasculature and traversing a region adjacent the cardiac valve.

3. The method according to claim 1, further comprising withdrawing the canalization-needle catheter after the suture is applied to the cardiac valve.

4. The method according to claim 1, wherein the suture comprises a cerclage suture.

5. The method according to claim 4, further comprising delivering a knot to the cerclage suture using a knot-delivery catheter.

6. The method according to claim 4, further comprising introducing tension to the cerclage suture to urge leaflets of the cardiac valve together.

7. The method according to claim 1, wherein the suture comprises a transverse-ligature portion.

8. The method according to claim 7, wherein the cardiac valve is the mitral valve, the suture extends at least partially through the coronary sinus, and the transverse-ligature portion extends from a posterolateral aspect of the coronary sinus to an anterior aspect of the coronary sinus.

9. The method according to claim 7, wherein the cardiac valve is the mitral valve, the suture extends at least partially through the coronary sinus, and the transverse-ligature portion extends from a septal aspect of the mitral-valve annulus to a lateral aspect of the mitral-valve annulus.

10. The method according to claim 1, wherein an imaging system is used to view the guiding catheter and the canalization-needle catheter in the vasculature of the subject.

11. A device for delivering a suture to a cardiac valve, comprising:
a flexible guiding catheter having a proximal end, a distal end, and a catheter lumen extending longitudinally through the guiding catheter, the guiding catheter being percutaneously insertable into the vasculature of a subject; and
a canalization-needle catheter having a proximal end and a distal end, the canalization-needle catheter being capable of sliding through the catheter lumen of the guiding catheter and extending beyond the distal end of the guiding catheter,

12. The device of claim 11, wherein the canalization-needle catheter is steerable.

13. The device of claim 11, wherein the canalization-needle catheter comprises a means for puncturing a vasculature wall.

14. The device of claim 11, wherein the canalization-needle catheter comprises a deflectable tip configured to penetrate and traverse through a vasculature wall in the subject.

15. The device of claim 11, wherein the suture is a cerclage suture.

16. The device of claim 11, wherein the guiding catheter further comprises a guide-wire lumen, and wherein the device further comprises a steerable guide wire along which the guide-wire lumen of the guiding catheter is capable of sliding.

17. The device of claim 16, wherein the guide wire comprises a portion adapted to puncture a vasculature wall.

18. The device of claim 16, wherein the guide wire comprises a deflectable tip configured to penetrate and traverse through a vasculature wall in a subject.

19. The device of claim 16, wherein the guide wire comprises a flexible tip having a moveable fulcrum.

20. The device of claim 11, wherein the guiding catheter and the canalization-needle catheter are manufactured at least partially of a material enhancing detectability of the guiding catheter and the canalization-needle catheter when they are viewed by an imaging system.

21. A device for applying a suture clip to a cardiac valve, comprising:
a flexible guiding catheter having a proximal end, a distal end, and a catheter lumen extending longitudinally through the guiding catheter, the guiding catheter being percutaneously insertable into the vasculature of a subject; and
a valve-manipulation catheter having a proximal end and a distal end, the valve-manipulation catheter being capable of sliding through the catheter lumen and extending beyond the distal end of the guiding catheter;
the valve manipulation catheter comprising a delivery system adapted to capture and apply a suture clip to a valve leaflet, wherein the delivery system comprises a spring loaded clip configured to grasp and apply a suture clip to the valve leaflet.
